(19) United States
(12) Patent Application Publication
(10) Pub. No.: US 2020/0381120 A1
(43) Pub. Date: Dec. 3, 2020

(54) IMPLANT IDENTIFICATION

(71) Applicant: Michael ISAACSON, Kirkland, WA (US)

(72) Inventor: Michael ISAACSON, Kirkland, WA (US)

(21) Appl. No.: 16/886,517

(22) Filed: May 28, 2020

Related U.S. Application Data
(60) Provisional application No. 62/855,730, filed on May 31, 2019.

(51) Int. Cl. G16H 50/20; G16H 10/20; G16H 70/00; G16H 30/20; G16H 30/40; G16H 40/40; G06Q 30/02; A61B 6/12; A61B 8/08; A61B 5/06; A61B 6/03; G06K 9/78; G06K 9/62

(52) U.S. Cl. CPC G16H 50/20; A61B 17/7032; G16H 70/00; G16H 30/20; G16H 30/40; G16H 40/40; G06Q 30/0215; A61B 6/12; A61B 8/0841; A61B 5/062; A61B 6/032; G06K 9/78; G06K 9/6215; G06K 9/6255; G16H 10/20

(57) ABSTRACT

An example system includes an image capture portion to provide an image of a medical implant; an identification portion coupled to the image capture portion; and a determination portion to facilitate identification of the medical implant, the determination portion including at least one of (a) a crowd source portion to survey a set of users, wherein results of the survey are provided to the identification portion; (b) a decision-based portion to perform decisions based on features of the image of the medical implant and to provide results of the decisions to the identification portion; or (c) a database-based portion to select information from a database of information related to medical implants, the selected information being determined to correspond to the image of the medical implant, wherein the selected information is to be provided to the identification portion.

This application claims the benefit of U.S. Provisional Patent Application No. 62/855,730, filed May 31, 2019, which is incorporated by reference herein in its entirety.

### BACKGROUND

Revision surgery is often performed for a variety of reasons. For example, in many cases, revision surgery may be performed to achieve improved results. In other cases, adjacent surgery may be performed to address issues proximate to an existing implant. For example, a successful implant provided at one spinal location may result in a weakness at an adjacent location, necessitating revision surgery at the adjacent location. In other contexts, revision surgery may be performed to correct an error made during the initial surgery. In some cases, revision surgery may include removal of a surgical implant.

### BRIEF DESCRIPTION OF THE DRAWINGS

For a more complete understanding of various examples, reference is now made to the following descriptions taken in connection with the accompanying drawings in which:

FIG. 1 illustrates an example system for identification of a surgical implant;

FIG. 2 illustrates an example image of an implant to be identified;

FIG. 3 is a flow chart illustrating an example method for identification of a surgical implant; and

FIGS. 4-6 are flow charts illustrating various example methods for identification illustrated in FIG. 3.

### DETAILED DESCRIPTION

As noted above, in certain cases, revision surgery may include removal of an implant. For example, a surgeon may wish to remove an old implant, such as a spinal implant, prior to addressing the patient's current problem. Such implants may include, for example, screws, rods, hooks, cervical plates, or the like. Implants are manufactured by numerous companies and often use proprietary locking mechanisms which require similarly proprietary tools (e.g., screwdrivers) for safe removal of the implant. Without the proper removal tools, the surgery may be difficult (e.g., requiring longer period of time) or even impossible. Identification of the implant and the necessary tools for removal of the implant is currently achieved in an ad hoc matter. Typically, a surgeon relies upon the availability of notes from the original surgeon, but such notes may or may not include sufficient detail to identify the implant or the necessary tool.

Various examples described herein provide systems and methods to facilitate identification of an implant. In various examples, an image of the implant may be captured using any of a variety of imaging mechanisms including, but not limited to, x-ray, digital x-ray, computed radiography (CR), digital radiography (DR), magnetic resonance imaging (MM), computed tomography (CT), ultrasound, or a combination of the various imaging mechanisms. The image of the implant may then be uploaded to an identification portion. In one example, the image may be shared by the identification portion with a crowd-source portion for surveying a set of users (e.g., crowd-source group members) to identify the implant. In another example, a decision-based portion performs a decision-based selection of the identity of the implant. As described in greater detail below, the decision-based selection may include the use of artificial intelligence or machine learning to facilitate identification of the implant. In still another example, the captured image of the implant(s) may be compared against a database of implants using, for example, artificial intelligence or machine learning. As the number of images are increased in the database, machine learning can help with the accuracy and speed of the database to improve confidence levels of matching images and implants in the system. Based on identification of the implant, the proper tool for removal of the implant may be identified.

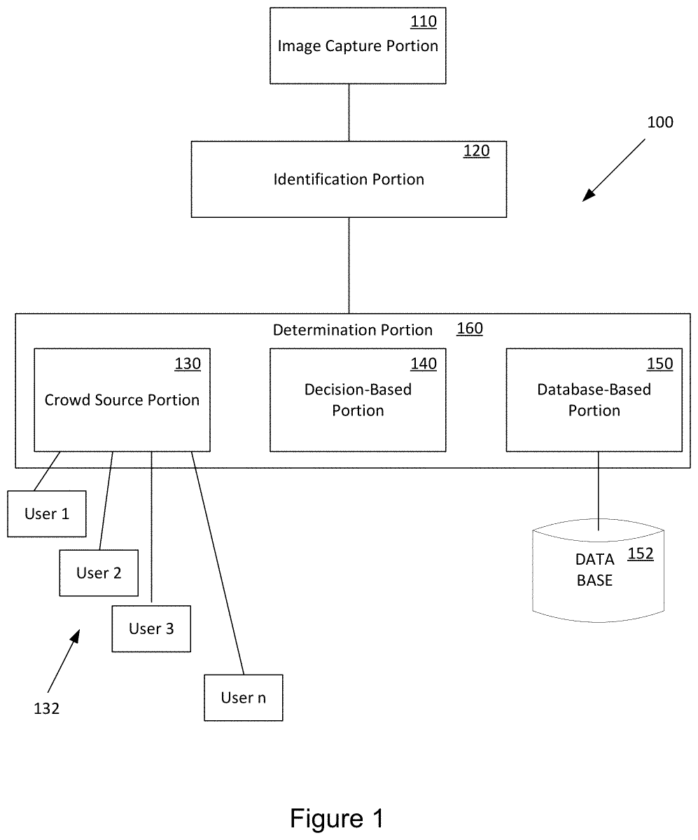

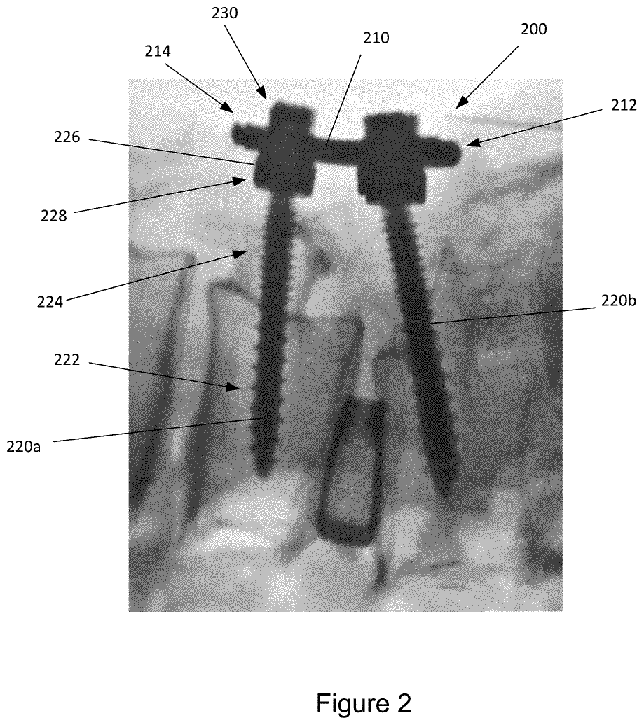

Referring now to the Figures, FIG. 1 illustrates an example system 100 for identification of a surgical implant. In various examples, the example system 100 may be a stand-alone system that may be accessible to users through, for example, a login portal. In other examples, the example system 100 may be accessed through a mobile application or a social media platform (e.g., Facebook™). The example system 100 includes an image capture portion 110. In various examples, the image capture portion 110 is provided to capture an image of the implant non-invasively. In this regard, the image capture portion 110 may include one or more of an x-ray, digital x-ray computed radiography (CR), digital radiography (DR), magnetic resonance imaging (MM), computed tomography (CT), ultrasound or a combination thereof, for example. The captured image may be saved in any of a variety of usable formats. An example of a captured image of an implant is illustrated in FIG. 2.

The example capture image illustrated in FIG. 2 may be captured using any of the imaging technologies noted above and includes the image of an implant 200. The implant 200 of FIG. 2 is characterized by various features. For example, the implant 200 includes a rod 210 that is secured to vertebrae by two screws 220a, 220b. In capturing the image of the implant 200, particular features of the implant can be noted. For example, the rod 210 may be noted as having a rounded end 212 on one end and a notched end 214 on the opposite end. The notched end 214 may be unique or uncommon for implants of this type and may serve as a key feature in identification of the implant 200.

Similarly, the screws 220a, 220b can be noted for particular features. In the example of FIG. 2, the screws 220a, 220b may be characterized as having two threaded regions which include a lower single-threaded region 222 and an upper double-threaded region 224. Additionally, the screw is provided with a tulip 226 which supports the rod 210 therein. The tulip 226 may be characterized as having a larger diameter 228 below the rod and a smaller diameter 230 above the rod. Each of the above-noted features of the implant 200 may be noted and used, either alone or in combination with other features, in identification of the implant 200.

Referring again to FIG. 1, the example system 100 further includes an identification portion 120. The identification portion 120 may be implemented as hardware, software, firmware or a combination thereof. In one example, the identification portion 120 is implemented in a processor. The identification portion 120 may be coupled to the image capture portion 110 to either receive or access the captured image of the implant.

The identification portion 120 may be coupled to a determination portion 160 which includes one or more portions to facilitate determination of the identity of an implant. In the example system 100 of FIG. 1, the identification portion 120 is coupled to the determination portion 160 which includes three portions, including a crowd source portion 130, a decision-based portion 140, and a database-based portion 150. Each portion 130, 140, 150 may operate independently or in conjunction with another portion 130, 140, 150. Thus, the identification portion 120 may use a determination of an implant by one or multiple portions 130, 140, 150 of the determination portion to identify the appropriate tool for removal of the implant.

The crowd source portion 130 of the example system 100 surveys a set of users 132. In this regard, the crowd source portion 130 can allow the set of users 132 to crowdsource and vote (or otherwise contribute) on the captured image to get a consensus on the implant manufacturer, implant system, and/or the proper instrumentation needed for removal of the implant. In this regard, the identification portion 120 may share the captured image with the set of users 132 through the crowd source portion 130 and provide a closed set of options from which the users 132 can vote. In some examples, a mechanism may be provided for the users 132 to write-in a different option or provide comments regarding the captured image. The set of users 132 may be made open to the general public or may be limited to a membership-based group. For example, membership may be limited to professionals in the medical and/or medical device community, including surgeons, medical device manufacturers, medical device sales persons, etc. In other examples, the set of users 132 may include healthcare providers, radiologists or other specialists. Based on the voting or other contributions of the set of users 132, an identity of an implant may be selected, and an associated tool may be identified for removal of the implant. The voting of the set of users 132 may be tabulated automatically or electronically by a processor. Comments or other contributions (e.g., write-in votes) may be reviewed by an administrator with electronic assistance. For example, comments may be categorized electronically and reviewed manually by the administrator. In some examples, the voting may result in a single candidate implant identification or a small number of candidate implant identifications from which a practitioner may select based on, for example, additional analysis of the physical implant or the patient's record. In some examples, members of the set of users 132 may be rewarded for voting or input which results in accurate identification of the implant. The reward may be financial or simply recognition of the contribution. Additionally, the amount of the reward (financial, points, status or other reward) may be varied based on the contribution of the member.

The decision-based portion 140 of the example system 100 allows for the identification of the implant using, for example, a self-directed decision algorithm. In one example, the decision algorithm may make decisions based on the location of the implant, the size of the implant and/or any of a variety of other features of the implant which may be identifiable with examination of the captured image. For example, for pedicle screws, the decision-making may be based on whether the screws have fixed or variable heads, top or side loading rods, fully threaded or smooth tip screw or other similar features. Similar decision-making may be provided for various categories of implants. Based on the results of the decision-making, a candidate identity of the implant may be presented to the user. In some examples, the candidate identity of the implant may be accompanied with a confidence level. For example, with each decision, the decision-based portion 140 may calculate a confidence level. The confidence level may be calculated based on a variety of factors, such as number of similar images in the database or affirmatively identified points of reference in images in the database.

In various examples, the decision-based portion 140 may include an artificial-intelligence, or machine learning, component. In this regard, with maturity of the system, the results may be accompanied with greater confidence levels.

The database-based portion 150 of the example system 100 is coupled to a database 152. The database 152 may include images and/or data associated with a variety of medical devices which may be used as implants. In one example, the database may include images of implants along with a corresponding identification. In this regard, the database-based portion 150 may perform an image comparison between the captured image and the various images in the database. In some examples, the database 152 may include synthetic images. Synthetic images may be generated by, for example, an artificial intelligence component, as described in greater detail below.

Synthetic images may be generated using, for example, a generative-adversarial network, or GAN. GANs combine a generative component and discriminative component and place them in adversarial positions. Discriminative components can categorize an instance of an image based on identified features. For example, an image of a medical implant may be categorized as either a medical implant or a non-medical implant or categorized as either a spinal implant or an implant for another part of the body.

While discriminative components categorize, or label, an instance based on features, a generative component can generate an instance based on a label or category. For example, for a category of spinal implants, the generative component may create a synthetic image with features associated with spinal implants.

In a GAN arrangement, the discriminative component may analyze real images of implants and associate features in the images with categories or labels. The discriminative component may perform a similar analysis on the synthetic images to attempt to discriminate between synthetic and real images. Thus, the generative component attempts to create synthetic images to trick the discriminative component into accepting them as real images, while the discriminative component attempts to identify the synthetic images to possible reject them as unacceptable. The synthetic images which are sufficiently realistic to trick the discriminative component may be added to the database.

In another example, the database-based portion 150 may perform an analysis of the captured image and extract information or data related to the implant. For example, the analysis of the captured image may yield various characteristics of the implant, such as size, type of fasteners, or color of the implant. In this example, the database 152 may be provided with similar data or information of various implants. Thus, in place of or in addition to the image comparison, the database may be queried for the data or information resulting from the analysis of the captured image. In one example, the database 152 may be supplemented or expanded with inputs from the crowd source portion 130. For example, images or information associated with the images obtained from crowdsourcing (e.g., from users 132) may be used to add images and/or information associated with the images to the database 152.

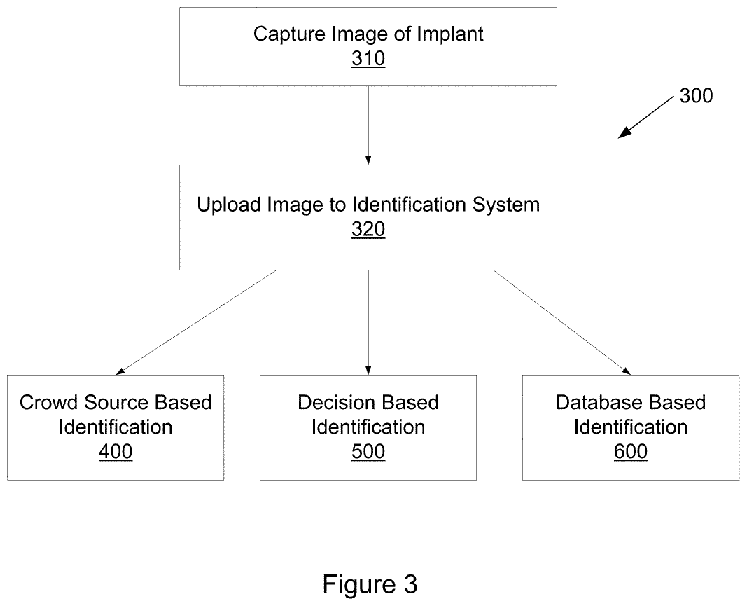

Referring now to FIG. 3, a flow chart illustrating an example method for identification of a surgical implant is provided. The example method 300 of FIG. 3 includes capturing an image of an implant (block 310). In one example, the capturing of the image may be performed by the image capture portion 110 described above with reference to FIG. 1. As noted above, the image may be captured using any of a variety of imaging techniques including, but not limited to, x-ray, CR, DR, MM, CT, or ultrasound. In some examples, the image capture portion 110 may search for and/or recognize patient identification information contained in the image. For privacy purposes, the image capture portion 110 may delete, blur or otherwise obscure the patient identification information from the image.

The captured image may be uploaded to an identification system (block 320). The identification system may include or be a part of the identification portion 120 described above with reference to FIG. 1. Uploading may include electronically transferring a digital representation of the captured image to the identification system, or a memory device associated with the identification system. The transfer may be initiated automatically upon capturing of the image or initiated manually by an operator. The identification system may be implemented in a processor, for example.

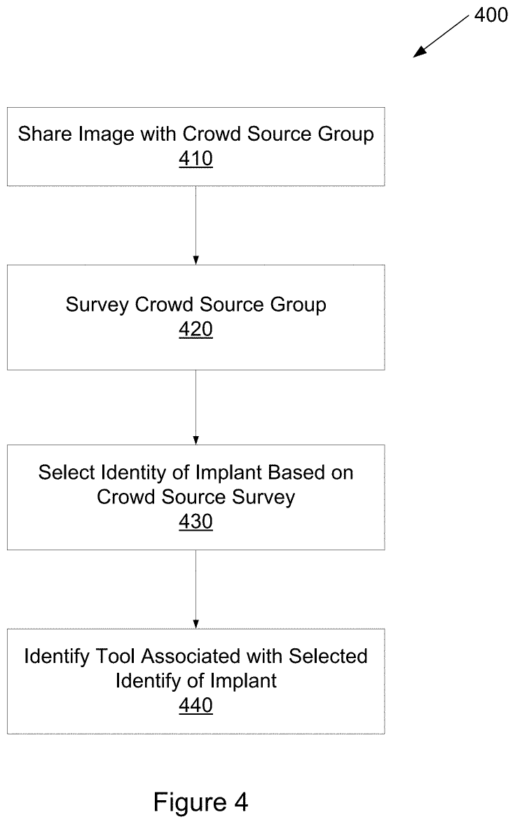

In various examples, the example method 300 may continue with identification of the implant using one or more of various identification mechanisms. The example method 300 of FIG. 3 is illustrated with three possible flow paths. In some examples, one of the available paths may be selected. In other examples, multiple paths may be selected to be performed either sequentially or in parallel. The three paths illustrated in FIG. 3 include using a crowd source based identification 400, a decision based identification 500 or a database based identification 600, each of which is described in greater detail below with reference to FIGS. 4-6.

Referring now to FIG. 4, a flow chart illustrates an example of the crowd source based identification 400 of FIG. 3. In one example, the crowd source based identification 400 may be executed by the crowd source portion 130 described above with reference to FIG. 1. The example method of FIG. 4 includes sharing the captured image with a crowd source group (block 410). The captured image may be shared by the crowd source group by posting the image to a web page, emailed to each member of the crowd source group, emailing a web link associated with the image to each member of the crowd source group, or by another similar mechanism. In some examples, prior to the sharing of the captured image, a determination may be made as to whether the captured image corresponds to a medical implant. If a determination is made that the captured image includes a medical implant, the example method 400 may be executed. As noted above, the crowd source group may be open to the general public or be limited to a specific group. The crowd source group is surveyed for input regarding the captured image (block 420). In this regard, the captured image may be presented to the crowd source group along with choices to be voted upon as to the identity of the implant in the captured image. As noted above, a mechanism may be provided to allow the crowd source group members to write in a choice not presented along with the image or to provide comments regarding the captured image.

The voting by the crowd source group may yield a consensus on the implant manufacturer, implant system, and/or the proper instrumentation needed for removal of the implant. Thus, based on the crowd source survey, an identity of the implant in the captured image may be selected (block 430). An associated tool may be selected based on the identification of the implant for removal of the implant (block 440). In some examples, once the implant, as well as the manufacturer, are identified and confirmed, a database of tools may be accessed to identify one or more tools for extraction of the implant. In this regard, multiple tools may be provided as options from which the practitioner may select. The list of multiple tools may be ordered from most appropriate (or best) to least appropriate (or worst) for the removal of the implant. For example, the best tool may be a tool manufactured by the manufacturer of the implant (e.g., a proprietary tool), while others may be standard tools (e.g., flathead, Phillips head, etc.).

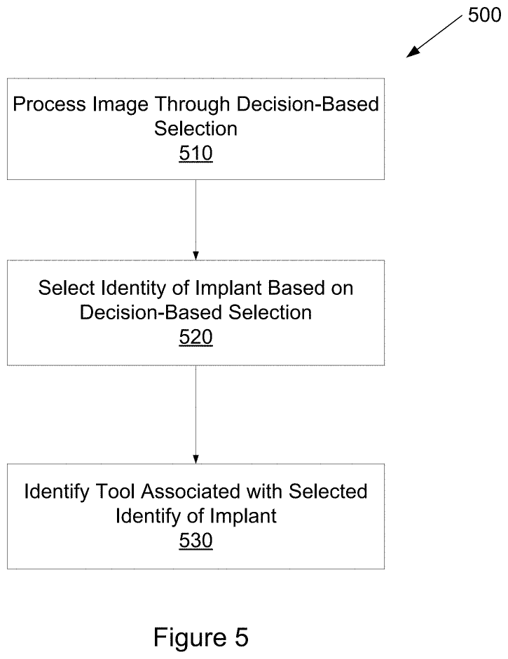

Referring now to FIG. 5, a flow chart illustrates an example of the decision based identification 500 of FIG. 2. In one example, the decision based identification 500 may be executed by the decision-based portion 140 described above with reference to FIG. 1. As illustrated in FIG. 5, the captured image of the implant may be processed through a decision-based selection (block 510). In various examples, the decision-based selection may include a self-directed decision algorithm. As described above, the decision algorithm may make decisions based on the location of the implant, the size of the implant and/or any of a variety of other features of the implant which may be identifiable with examination of the captured image. Based on the results of the decision-making, an identity of the implant may be selected (block 520) and presented to the user. In some examples, the identity of the implant may be accompanied with a confidence level. In various examples, the decision-based portion 140 may include an artificial-intelligence, or machine learning, component. In this regard, with maturity of the system, the results may be accompanied with greater confidence levels. An associated tool may be selected based on the identification of the implant for removal of the implant (block 530).

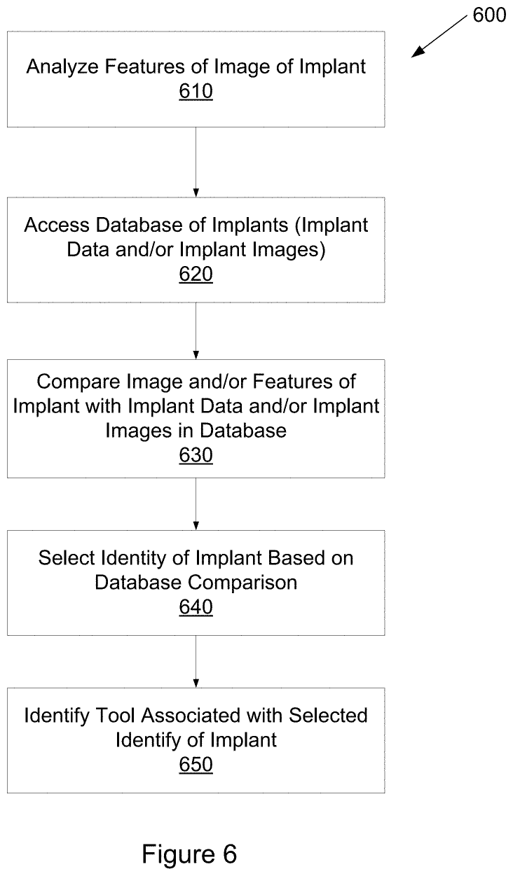

Referring now to FIG. 6, a flow chart illustrates an example of the database based identification 600 of FIG. 3. In one example, the database based identification 600 may be executed in the database-based portion 150 described above with reference to FIG. 1. As illustrated in FIG. 6, the example method 600 includes analyzing features of the implant (block 610). In some examples, the features of the implant may be extracted or identified from the captured image of the implant. In other examples, the features of the implant may be obtained from various other sources, such as surgical notes from the previous surgery, for example. In this regard, the surgical notes may be provided in digital form and may be accessed through an electronic medical record. For example, the example system 100 described above with reference to FIG. 1 may access the electronic medical record using an application program interface (API) corresponding to the electronic medical record. Text recognition may be performed, and natural language processing may be used to obtain textual features from the surgical notes in the medical record related to the implant. In one example, various features of the implant (e.g., from the captured image) may be noted, such as the location, size, color or any of a variety of other parameters related to the implant in the captured image. In other examples, textual features obtained from, for example, the surgical record may include, without limitation, the date of the previous surgery, name of the surgeon or hospital, name of the manufacturer of the implant, system or brand name of the implant, size or location of the implant, or a category type of implant and/or fastener (e.g., multiaxial screw, percutaneous, translational cervical plate, titanium interbody cage, etc.).

A database of implants may be accessed (block 620). As noted above, the database, such as the database 152 described above with reference to FIG. 1, may include images and/or data associated with a variety of medical devices which may be used as implants. In one example, the database may include images of implants along with a corresponding identification. In another example, the database-based portion 150 may perform an analysis of the captured image and extract information or data related to the implant. For example, the analysis of the captured image may yield various characteristics of the implant, such as size, type of fasteners, or color of the implant.

The captured image of the implant and/or various features of the implant extracted from the captured image may be compared against the images or the information in the database. For example, an image comparison between the captured image and the various images in the database may be performed, or the database may be queried for data or information resulting from the analysis of the captured image. Based on the comparison, an identity of the implant in the captured image may be selected (block 640), and an associated tool may be selected based on the identification of the implant for removal of the implant (block 650).

In each of the examples described above in FIGS. 4-6, following identification of the appropriate tool (e.g., blocks 440, 530, or 650), the system may form a connection with the manufacturer or seller of the appropriate tool through, for example, a website, a mobile application, email, phone or another point of sale. Through this connection, the manufacturer or seller may be notified of the desire or need to obtain the tool and may be provided with a delivery address. Further, the connection may be used to verify the identification of the implant and the tool.

In some examples, the results of the example method 300 for identification of a surgical implant, the crowd source based identification 400, the decision based identification 500, the database based identification 600, or a combination thereof may be integrated into a pre-surgery plan. For example, the identification of the implant and/or an associated tool can be provided to or integrated with the pre-surgery plan through a pre-surgery planning software. In this regard, the example system 300 described above with reference to FIG. 3 may be coupled to or integrated with the pre-surgery planning software through appropriate application programming interfaces (APIs).

Thus, identification of an implant may be facilitated prior to revision surgery. The identification may provide the information needed to obtain the proper tools for effective removal of the implant during the revision surgery and incorporated into a pre-surgery plan.

Software implementations of various examples can be accomplished with standard programming techniques with rule-based logic and other logic to accomplish various database searching steps or processes, correlation steps or processes, comparison steps or processes and decision steps or processes.

The foregoing description of various examples has been presented for purposes of illustration and description. The foregoing description is not intended to be exhaustive or limiting to the examples disclosed, and modifications and variations are possible in light of the above teachings or may be acquired from practice of various examples. The examples discussed herein were chosen and described in order to explain the principles and the nature of various examples of the present disclosure and its practical application to enable one skilled in the art to utilize the present disclosure in various examples and with various modifications as are suited to the particular use contemplated. The features of the examples described herein may be combined in all possible combinations of methods, apparatus, modules, systems, and computer program products.

It is also noted herein that while the above describes examples, these descriptions should not be viewed in a limiting sense. Rather, there are several variations and modifications which may be made without departing from the scope as defined in the appended claims.

What is claimed is:

1. A system, comprising:
an image capture portion to provide an image of a medical implant;
an identification portion coupled to the image capture portion; and
a determination portion to facilitate identification of the medical implant, the determination portion including at least one of:
(a) a crowd source portion to survey a set of users, wherein results of the survey are provided to the identification portion;
(b) a decision-based portion to perform decisions based on features of the image of the medical implant and to provide results of the decisions to the identification portion; or
(c) a database-based portion to select information from a database of information related to medical implants, the selected information being determined to correspond to the image of the medical implant, wherein the selected information is to be provided to the identification portion.

2. The system of claim 1, wherein the identification portion is provided to facilitate identification of a tool associate with the medical implant based on identification of the medical implant by the identification portion.

3. The system of claim 1, wherein the crowd source portion receives votes or comments from the set of users.

4. The system of claim 3, wherein the crowd source portion provides a single candidate implant identification or multiple candidate implant identifications.

5. The system of claim 1, wherein the crowd source portion provides a reward to one or more members of the set of users based on survey input.

6. The system of claim 1, wherein the set of user of the crowd source portion is limited to professionals in a medical or medical device community.

7. The system of claim 1, wherein the results provided by the decision-based portion include at least one candidate identity of the medical implant and a corresponding confidence level.

8. The system of claim 7, wherein the confidence level is based on at least one of a number of matches to similar images in a database or a number of matching points of reference.

9. The system of claim 1, wherein the database of information of the database-based portion includes images of medical implants.

10. The system of claim 9, wherein the database-based portion includes an artificial intelligence component to generate synthetic images to be added to the database of information.

11. The system of claim 1, wherein the image capture portion includes at least one of x-ray, digital x-ray, computed radiography (CR), digital radiography (DR), magnetic resonance imaging (MRI), computed tomography (CT), or ultrasound.

12. A method, comprising:
capturing image of a medical implant;
uploading the image to an identification system; and
determining candidate identities of the medical implant, wherein determining the candidate identities includes at least one of the following:
(a) performing a crowd-source based identification, comprising conducting a survey of a set of users, wherein results of the survey are provided to the identification portion;
(b) performing a decision-based identification, comprising making decisions based on features of the image of the medical implant and providing results of the decisions to the identification portion; or
(c) performing a database-based identification, comprising selecting information from a database of information related to medical implants, the selected information being determined to correspond to the image of the medical implant, wherein the selected information is to be provided to the identification portion.

13. The method of claim 12, further comprising:
using the identification system to facilitate identification of a tool associated with the medical implant based on determining the candidate identities.

14. The method of claim 12, wherein performing the crowd-source based identification includes receiving votes or comments from the set of users.

15. The method of claim 12, further comprising:
providing a reward to one or more members of the set of users based on survey input in the crowd-source based identification.

16. The method of claim 12, wherein the set of user in the crowd-source based identification is limited to professionals in a medical or medical device community.

17. The method of claim 12, wherein the results from the decision-based identification include at least one candidate identity of the medical implant and a corresponding confidence level.

18. The method of claim 17, wherein the confidence level is based on at least one of a number of matches to similar images in a database or a number of matching points of reference.

19. The method of claim 1, wherein the database of information used in the database-based identification includes images of medical implants.

20. The method of claim 19, wherein performing the database-based identification includes executing an artificial intelligence component to generate synthetic images to be added to the database of information.
